SEQUENCE LISTING (2 sequences)

SEQ ID NO: 1 - skipped

SEQ ID NO: 2 - protein, 2415 aa, Homo sapiens
MTTLLWVFVTLRVITAAVTVETSDHDNSLSVSIPQPSPLRVLLGTSLTIPCYFIDPMHPVTTAPSTAPLAPRIKWSRVSKEKEVVLLVATEGRVRVNSAYQDKVSLPNYPAIPSDATLEVQSLRSNDSGVYRCEVMHGIEDSEATLEVVVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGHVYLAWQAGMDMCSAGWLADRSVRYPISKARPNCGGNLLGVRTVYVHANQTGYPDPSSRYDAICYTGEDFVDIPENFFGVGGEEDITVQTVTWPDMELPLPRNITEGEARGSVILTVKPIFEVSPSPLEPEEPFTFAPEIGATAFAEVENETGEATRPWGFPTPGLGPATAFTSEDLVVQVTAVPGQPHLPGGVVFHYRPGPTRYSLTFEEAQQACPGTGAVIASPEQLQAAYEAGYEQCDAGWLRDQTVRYPIVSPRTPCVGDKDSSPGVRTYGVRPSTETYDVYCFVDRLEGEVFFATRLEQFTFQEALEFCESHNATATTGQLYAAWSRGLDKCYAGWLADGSLRYPIVTPRPACGGDKPGVRTVYLYPNQTGLPDPLSRHHAFCFRGISAVPSPGEEEGGTPTSPSGVEEWIVTQVVPGVAAVPVEEETTAVPSGETTAILEFTTEPENQTEWEPAYTPVGTSPLPGILPTWPPTGAETEESTEGPSATEVPSASEEPSPSEVPFPSEEPSPSEEPFPSVRPFPSVELFPSEEPFPSKEPSPSEEPSASEEPYTPSPPEPSWTELPSSGEESGAPDVSGDFTGSGDVSGHLDFSGQLSGDRASGLPSGDLDSSGLTSTVGSGLTVESGLPSGDEERIEWPSTPTVGELPSGAEILEGSASGVGDLSGLPSGEVLETSASGVGDLSGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETTAPGVEEISGLPSGEVLETTAPGVDEISGLPSGEVLETTAPGVEEISGLPSGEVLETSTSAVGDLSGLPSGGEVLEISVSGVEDISGLPSGEVVETSASGIEDVSELPSGEGLETSASGVEDLSRLPSGEEVLEISASGFGDLSGVPSGGEGLETSASEVGTDLSGLPSGREGLETSASGAEDLSGLPSGKEDLVGSASGDLDLGKLPSGTLGSGQAPETSGLPSGFSGEYSGVDLGSGPPSGLPDFSGLPSGFPTVSLVDSTLVEVVTASTASELEGRGTIGISGAGEISGLPSSELDISGRASGLPSGTELSGQASGSPDVSGEIPGLFGVSGQPSGFPDTSGETSGVTELSGLSSGQPGVSGEASGVLYGTSQPFGITDLSGETSGVPDLSGQPSGLPGFSGATSGVPDLVSGTTSGSGESSGITFVDTSLVEVAPTTFKEEEGLGSVELSGLPSGEADLSGKSGMVDVSGQFSGTVDSSGFTSQTPEFSGLPSGIAEVSGESSRAEIGSSLPSGAYYGSGTPSSFPTVSLVDRTLVESVTQAPTAQEAGEGPSGILELSGAHSGAPDMSGEHSGFLDLSGLQSGLIEPSGEPPGTPYFSGDFASTTNVSGESSVAMGTSGEASGLPEVTLITSEFVEGVTEPTISQELGQRPPVTHTPQLFESSGKVSTAGDISGATPVLPGSGVEVSSVPESSSETSAYPEAGFGASAAPEASREDSGSPDLSETTSAFHEANLERSSGLGVSGSTLTFQEGEASAAPEVSGESTTTSDVGTEAPGLPSATPTASGDRTEISGDLSGHTSQLGVVISTSIPESEWTQQTQRPAETHLEIESSSLLYSGEETHTVETATSPTDASIPASPEWKRESESTAAAPARSCAEEPCGAGTCKETEGHVICLCPPGYTGEHCNIDQEVCEEGWNKYQGHCYRHFPDRETWVDAERRCREQQSHLSSIVTPEEQEFVNNNAQDYQWIGLNDRTIEGDFRWSDGHPMQFENWRPNQPDNFFAAGEDCVVMIWHEKGEWNDVPCNYHLPFTCKKGTVACGEPPVVEHARTFGQKKDRYEINSLVRYQCTEGFVQRHMPTIRCQPSGHWEEPRITCTDATTYKRRLQKRSSRHPRRSRPSTAH

(19) United States
(12) Patent Application Publication
(10) Pub. No.: US 2009/0149332 A1
(43) Pub. Date: Jun. 11, 2009

(54) METHODS FOR DETERMINING THE ACTIVITY OF ADAM-TS PROTEASES USING THIOPEPTOLIDES

(75) Inventors: Klaus-Ulrich Weithmann, Hofheim (DE); Volker Jeske, Glashutten (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 11/814,936

(22) PCT Filed: Feb. 10, 2006
(86) PCT No.: PCT/EP06/01184

§ 371 Date: Sep. 5, 2008

(30) Foreign Application Priority Data
Feb. 11, 2005 (DE) 10 2005 005 972.4

(51) Int. Cl. C40B 30/00; C12Q 1/37

(52) U.S. Cl. 506/7; 435/23

(57) ABSTRACT

The invention concerns the use of thiopeptolides of formula R-(Xaa)ₙ-Pro-X-Gly-S—Y-Z-Gly-(Xaa)ₘ-R₁ (I) as substrate for determining the activity of ADAMTS proteases, and a method for finding an ADAMTS protease modulator, more particularly an inhibitor.

The present invention relates to the use of thiopeptolides of the formula R-(Xaa)ₙ-Pro-X-Gly-S—Y-Z-Gly-(Xaa)ₘ-R₁ (I) as substrate for determining the activity of ADAM-TS proteases and to a method for finding an ADAM-TS protease modulator, in particular an inhibitor.

An intact articular cartilage matrix is the decisive prerequisite for the functioning of all joints of the animal and human body. Damage to the articular cartilage leads to arthritic diseases such as osteoarthrosis (osteoarthritis) and rheumatism, which are characterized by dysfunction and finally immobility of the affected animal or human.

Among further diseases characterized by impaired matrix degradation must also be included the diverse forms of cancers, especially the metastasis of tumors.

It has been known for some time that matrix metalloproteases (MMPs) are involved in the degradation of the aggrecan and collagen in cartilage. These include for example the group of matrixins, which comprises all known MMPs from MMP-1, 2 etc, up to MMP-16. A further group, namely the proteases of the ADAM-TS-family (Nagase, H. et al. (2003), Arthritis Research Therapy, 5, 94-103), likewise plays a crucial role in the degradation of tissue matrix, resulting in damage to the cartilage matrix. The activity of these proteases, especially ADAM-TS 1, ADAM-TS 4 and ADAM-TS 5, which are also referred to as ‘aggrecanases’, is the cause of diseases characterized by impaired matrix degradation, such as osteoarthrosis, rheumatism and cancer. ADAM-TS is able to cleave in particular proteoglycan, but also other matrix constituents such as hyaluronan or collagen. Further effects of ADAM-TS 1, 4, 5 and 11, but also ADAM-TS 13, are crucial in inflammatory processes, angiogenesis, cell migration and blood clotting, or blood coagulation (Apte, S. S. (2004), The international Journal of Biochemistry & Cell Biology, 36, 981-985.

It is therefore an important task of pharmaceutical research on the one hand to be able to detect the enzymatic activity of the proteases involved in the diseases in the tissue at risk or already diseased, e.g. cartilage tissue, or blood. However, on the other hand, it is also particularly important to develop pharmaceuticals able to inhibit single, a plurality of, or all relevant proteases.

The enzymatic (proteolytic) activity of the proteases involved can be measured in vitro by incubating the relevant protease with the appropriate high molecular weight matrix components, e.g. proteoglycan or collagen, and measuring the formation of the degradation products.

Various methods which regularly make elaborate procedures necessary, such as antibody recognition of specific cleavage sites, and mass spectrometric investigations, are available to the skilled worker for isolating and quantifying the heterogeneous degradation products, that is to say for example collagen fragments and protein fragments.

It has previously been described for example that a recombinant substrate which comprises important structural elements of the interglobular domain of natural aggrecan can be used to determine the activity of ADAM-TS 4. The recombinant aggrecan of molecular weight 72 kDa is expressed in COS cells. Determination of the aggrecanase activity requires, besides the use of the high molecular weight aggrecan molecule, further elaborate steps such as structural elements which the signal sequence of CD5, the FLAG epitope for the M1 monoclonal antibody determination, the hinge region of human IgG1, cDNA for the recombinant substrate mentioned, including vectors thereof, as described in detail in EP 0785 274 and also in Hörber, Chr. et al. (2000) Matrix Biology, 19, 533-543.

The use of shorter fragments of the aggrecan molecule has also been disclosed, WO 00/05256 reported that these peptide fragments may consist of 20 to 40 amino acid building blocks. However, it is particularly disadvantageous that peptides comprising fewer than 20 amino acids cannot be converted with aggrecanase. It has been possible to confirm this (see experimental section).

To determine the proteases of the matrixin family, according to the prior art low molecular weight molecules which can be obtained easily by synthesis are cleaved as substrates of the proteases to determine the enzymatic (proteolytic) activity, there being, because of the particular nature of these substrates, release by cleavage of, for example, an optical signal, ordinarily in the visible or ultraviolet wavelength range, which can be quantified.

A well-known example is (7-methoxycoumarin-4-yl)acetyl-Pro-Leu-Gly-Leu-3-(2′,4′-dinitro-phenyl)-L-2,3-diaminopropionyl-Ala-Arg-NH₂ (Bachem, Heidelberg, Germany) which was described by Knight, C. G. et al. (1992) FEBS, 296, 263-266 and which is cleaved by particular matrix metalloproteinases, and thus releases a measurable fluorimetric signal which can be used to calculate the enzymic activity. It has thus been possible to confirm that it is possible to convert (7-methoxycoumarin-4-yl)acetyl-Pro-Leu-Gly-Leu-3-(2′,4′-dinitrophenyl)-L-2,3-diamino-propionyl-Ala-Arg-NH₂ with the proteases MMP-3 and MMP-8 (see experimental section). It was also possible to convert many further substrates of this type by MMP-3 and MMP-8.

It has also been disclosed that certain substrates from the thiopeptolide class of substances are converted by collagenase and membrane-associated matrix metalloproteinases, of EP 0149593 and US 2002/0142362. However, aggrecanase cleavage of a peptide truncated to 16 amino acids N-terminally up to the aggrecanase cleavage site no longer occurs.

It was also possible to confirm in some experiments that shorter-chain peptides, even if they comprise the sequence Glu-Ala, could not be cleaved by ADAM-TS. By contrast, it has been disclosed that the members of other protease families, e.g. the matrixins or the cathepsins, are able to cleave oligopeptides.

The use of oligopeptides in such experiments is particularly desired because they are easily obtainable by chemical synthesis. A further advantage of oligopeptides is that individual peptide building blocks can be chemically modified, e.g. can also be linked to those chromophoric groups which permit cleavage of the peptide to be followed directly or indirectly by spectrometry, e.g. colorimetry.

In a corresponding manner it is also possible for the effect of enzyme inhibitors or activators easily to be determined by comparing the proteolytic activity of the relevant protease after addition of the inhibitor with the activity measured before addition of the inhibitor.

One example thereof are the thiopeptolides R-Pro-X-Gly-S—Y-Z-Gly-R1 (EP 0149593), which are cleaved by vertebrate collagenase, and acetyl-prolyl-leucyl-glycyl-[2 mercapto4-methyl-pentanoyl]-leucyl-glycyl-ethyl ester (US 2002/0142362), which is cleaved by certain matrixins, thus forming a free SH group which can be quantified by known methods, e.g. reaction with DTNB.

It has now been found according to the invention that the thiopeptolides R-(Xaa)ₙ-Pro-X-Gly-S—Y-Z-Gly-(Xaa)ₘ-R₁ of the formula (I) are cleaved by ADAM-TS proteases, in particular by ADAM-TS1, ADAM-TS4, ADAM-TS5, ADAM-TS11 and/or ADAM-TS13, especially by ADAM-TS1, ADAM-TS4 or ADAM-TS5, and can thus easily be detected via the free SH group. This is all the more surprising since the prior art reports that peptides comprising fewer than 16 amino acid units cannot be cleaved by ADAM-TS.

One aspect of the present invention therefore relates to the use of a thiopeptolide of the formula

R-(Xaa)ₙ-Pro-X-Gly-S—Y-Z-Gly-(Xaa)ₘ-R₁ (I),
where
   R is H or an N-protective group, preferably a carboxyl group, in particular of C₁-C₅-alkyls, especially of C₁-C₃-alkyls, particularly preferably an acetyl group,
   Xaa=any naturally occurring amino acid,
   n, m=identically or differently an integer from 0-2415, preferably from 0-35, in particular from 0-14, especially from 0-11 and very particularly preferably equal to 0,
   X=Leu, Ile, Phe, Val, Gln, Ala,
   Z=Leu, Ile, Phe, Val, Gln, Ala,
   R₁=terminal amide, carboxyl or ester group, preferably of C₁-C₅-alkyls, especially of C₁-C₃-alkyls, in particular an ethyl ester,

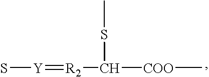

where
   R₂ is the side chain of a naturally occurring amino acid, in particular
   —CH₂CH(CH₃)₂,
   —CH(CH₃)C₂H₅,
   —CH₂C₆H₅,
   —CH(CH₃)₂, or
   —CH₃,
      or a salt thereof, as a substrate for an ADAM-TS protease.

Suitable N-protective groups are generally all conventional amino acid protective groups such as, for example, Fmoc (9-fluoroenylmethyloxycarbonyl), Mtt (4-methyltrityl), Pmc (2,2,5,7,8-pentamethylchroman-6-sulfonyl), tBu (t-butyl), Boc (t-butyloxycarbonyl), Tos (tosyl), Mbzl (4-methylbenzyl), Bom (benzyloxymethyl), 2-chloro-Z (2-chlorobenzyloxycarbonyl) or For (formyl), as can be obtained for example from Bachem Distribution Services GmbH, Weil am Rhein.

A particularly preferred thiopeptolide of the formula (I) is one in which
X=Leu or Ala,
Z=Leu, Ala or Phe, and
R₂=—CH₂CH(CH₃)₂.

In another preferred embodiment, (Xaa)ₙ and/or (Xaa)ₘ, corresponds to the amino acid sequence shown in SEQ ID NO: 2, which represents the amino acid sequence for human aggrecan.

Further preferred thiopeptolides have the following structure:

Ac-Pro-Leu-Gly-S—Y-Leu-Gly-OC₂H₅,
in which R₂=CH₂CH(CH₃)₂, and Ac is generally an acetyl group,
   or

Ac-Pro-Leu-Gly-S—Y-Phe-Gly-OC₂H₅,
in which R₂=CH₂CH(CH₃)₂,
   or

Ac-Pro-Ala-Gly-S—Y-Phe-Gly-OC₂H₅,
in which R₂=CH₂CH(CH₃)₂,
   or

Ac-Pro-Ala-Gly-S—Y-Ala-Gly-OC₂H₅,
in which R₂=CH₂CH(CH₃)₂.

All known ADAM-TS proteases are suitable according to the present invention, such as the ADAM-TS protease 1, 2, 3, 4, 5, 6, 7, 8, 9, 10, 11, 12, 13, 14, 15, 16, 17, 18, 19 and/or 20. The ADAM-TS proteases 1, 4, 5, 11 and/or 13 are preferred, especially the ADAM-TS protease 1, ADAM-TS protease 4 or ADAM-TS protease 5.

The use according to the invention is particularly suitable for determining the activity of an ADAM-TS protease, for purifying an ADAM-TS protease, for functional cloning of a nucleotide sequence coding for an ADAM-TS protease, for finding an ADAM-TS protease modulator, in particular an ADAM-TS protease inhibitor, or for observing the onset or progress of a disease associated with damaged tissue matrix, in particular osteoarthritis, rheumatism, cancer, inflammations, angiogenesis, cell migration, blood clotting and/or blood coagulation, especially osteoarthritis, rheumatism or cancer. In all these methods, e.g. during the enzymatic purification of an ADAM-TS protease or after cloning of a gene coding for an ADAM-TS protease into a generally known expression vector, ultimately the activity of the ADAM-TS protease is measured with the aid of the described thiopeptolides.

The present invention therefore also relates to a method for determining the activity of an ADAM-TS protease, where the method comprises the following steps:
(a) incubating an ADAM-TS protease with a thiopeptolide substrate according to formula I or as described above in detail, and
(b) carrying out an activity measurement or determination of the ADAM-TS protease.

Suitable and preferred ADAM-TS proteases are the ADAM-TS proteases which have been described in detail above. The activity measurement or determination is preferably carried out by spectrophotometry.

In the determination by spectrophotometry there is usually employment of a detection reagent, in this case for thiol groups. Those which have proved advantageous in this connection are 4,4′-dithiodipyridine or, in particular, 5,5′-dithiobis(2-nitrobenzoic acid) (DTNB), which is also known as Ellmann's reagent. However, it is also possible to employ any other suitable thiol-reactive reagents such as an iodoacetamide, e.g. 5-iodoacetamidofluorescein (5-IAF), a maleimide, e.g. fluorescein-5-maleimide, or other thiol-reactive reagents such as N,N′-didansyl-L-cystine or 5-(bromomethyl)fluorescein. Reagents of these types can be obtained for example from invitrogen GmbH, Karlsruhe.

It is possible with the aid of said thiopeptolides to find in a suitable assay system particularly simply ADAM-TS protease modulators. Modulators mean according to the present invention in particular ADAM-TS protease activators and especially ADAM-TS protease inhibitors.

A further aspect of the present invention therefore relates to a method for finding an ADAM-TS protease modulator, in particular an ADAM-TS protease inhibitor, in which the method comprises the following steps:
(a) incubating an ADAM-TS protease with a thiopeptolide substrate according to formula I or as described in detail above in the presence of a test compound and
(b) measuring or determining the influence of the test compound on the activity of the ADAM-TS protease.

Suitable and preferred ADAM-TS proteases are the ADAM-TS proteases which have been described in detail above. The activity measurement or determination is preferably carried out by spectrophotometry as already described in detail above. The compounds described above are in turn suitable as detection reagent for thiol groups, especially 4,4′-dithiodipyridine or 5,5′-dithiobis(2-nitrobenzoic acid) (DTNB).

The test compound may be any conceivable chemical, biochemically, naturally occurring or synthetic, high or low molecular weight compound. It is particularly advantageous for the test compounds to be made available in the form of a chemical compound library from which the desired compound, e.g. an inhibitor of the tested ADAM-TS protease, can then be found with the aid of the method of the invention.

In a further preferred embodiment, the method of the invention is carried out on an array which particularly facilitates the finding and isolation of the desired compound. The use of a robot for carrying out the method of the invention likewise leads to a further facilitation and to an increase in the throughput and is therefore particularly advantageous. It is also possible with the aid of microfluidic technology, where appropriate combined with miniaturized plate recesses (“wells”), to miniaturize and further automate the assay system, which is in turn particularly advantageous. The method is generally employed in a high-throughput screening for an ADAM-TS protease modulator, in particular an ADAM-TS protease inhibitor.

Another aspect of the present invention, which is based on the method of the invention, relates to the manufacture of a medicament, which comprises the following steps:
(a) carrying out the abovementioned method for finding an ADAM-TS modulator, in particular an inhibitor,
(b) isolating a test substance found to be suitable in step (a), and
(c) formulating the test substance isolated in step (b) with one or more pharmaceutically acceptable carriers or adjuvants.

The pharmaceutically active compounds, preferably inhibitors, found with the aid of the method of the invention are particularly suitable for the treatment of a disease which is associated with damaged tissue matrix, in particular osteoarthritis, rheumatism, cancer, inflammations, angiogenesis, cell migration, blood clotting and/or blood coagulation, in particular osteoarthritis, rheumatism or cancer.

All known agents which are normally employed for pharmaceutical formulation are suitable as pharmaceutically acceptable carrier or adjuvant.

Examples are a sodium chloride solution, in particular an isotonic saline solution (0.9% strength sodium chloride solution), demineralized water, stabilizers such as protease inhibitors or nuclease inhibitors, preferably aprotinin, s-aminocaproic acid or pepstatin A, or masking agents such as EDTA, gel formulations such as white petrolatum, low-viscosity paraffin and/or yellow wax, depending on the mode of administration.

Further suitable additives are for example detergents such as triton X-100 or sodium deoxycholate, but also polyols such as polyethylene glycol or glycerol, sugars such as sucrose or glucose, zwitterionic compounds such as amino acids, e.g. glycine or, in particular, taurine or betaine and/or a protein such as bovine serum albumin or human serum albumin. Detergents, polyols and/or zwitterionic compounds are particularly preferred.

The physiological buffer solution preferably has a pH of approximately 6.0-8.0, in particular a pH of approximately 6.8-7.8, especially a pH of approximately 7.4 and/or an osmolarity of approximately 200-400 milliosmole/liter, preferably approximately 290-310 milliosmole/liter. The pH of the medicament is generally adjusted with the aid of suitable organic or inorganic buffers, e.g. preferably with the aid of phosphate buffer, tris-buffer (tris(hydroxymethyl)aminomethane), HEPES buffer ([4-(2-hydroxyethyl)piperazino]ethanesulphonic acid) or MOPS buffer (3-morpholino-1-propanesulphonic acid). Choice of the appropriate buffer generally depends on the desired buffer molarity. Phosphate buffer is suitable for example for solutions for injection and infusion.

A further aspect of the present invention is a kit which is based on the thiopeptolide substrate of the invention according to formula I or as described above in detail and an ADAM-TS protease as described above and comprises where appropriate one or more buffers, e.g. TNCB buffer (see example). The buffer serves as stabilizing or reaction medium for carrying out the method of the invention. The kit preferably comprises in addition a detection reagent for thiol groups, e.g. at least one of the detection reagents described above, in particular 4,4′-dithiodipyridine and/or 5,5′-dithiobis(2-nitrobenzoic acid) (DTNB). A further component of the kit may be instructions for use for carrying out the method of the invention, in particular the activity assay.

The following statements and examples are intended to explain the invention in detail without restricting it thereto:

### Sequence Listing

SEQ ID NO: 1 corresponds to the thiopeptolide according to formula (I)

SEQ ID NO: 2 represents the amino acid sequence of the aggrecan core protein precursor (cartilage-specific proteoglycan core protein, CSPCP or chondroitin sulfates proteoglycan core protein 1).

### EXAMPLES

### Example 1

### Comparative Example

### Assay Conditions

The catalytic domains of the human recombinant MMP-3 and MMP-8 protein, MMP-3cd and MMP-8cd, can be purchased from, for example, Biomol International L. P. Pennsylvania, USA; catalog number SE-109 and SE-255 respectively. The truncated forms of ADAM-TS1 and ADAM-TS4 protease can likewise be purchased from, for example, Invitek, Gesellschaft für Biotechnik & Biodesign mbH, Berlin, Germany, catalog number 30400402 and 30400102, respectively.

### Preparation of the Buffers and Solutions

### TNCB Buffer:

100 mM tris(hydroxymethyl)aminomethane, adjusted to pH 7.5 with HCl-100

### 100 mM NaCl

### 10 mM CaCl₂.2H₂O

### 0.015% Brij 35

### Substrate Solution:

10 mM substrates (see Table) in DMSO, immediately before use, 2 μL of the substrate stock solution were diluted with 130 μL of H₂O.

### Enzyme Solution:

MMP-3cd (2.3 μg/mL), MMP-8cd (0.6 μg/mL), ADAMTS-1 (2.3 μg/mL) and ADAMTS-4 (3.3 μg/mL) were diluted with TNCB buffer.

Assay Procedure

10 μL of enzyme solution were mixed with 10 μL of H₂O, and the reaction was started by adding 10 μL of substrate solution.

### Fluorometric Analysis

The fluorescence was measured in a TECAN Spectrafluor Plus fluorescence apparatus; Excitation/Emission (see Table). This entailed measuring the fluorescence for 5 minutes on each occasion.

### Results of Comparative Example 1

| | | | ADAMTS- | ADAMTS- | MMP- | MMP- |
|---|---|---|---|---|---|---|
| Fluorimetric substance | | λₑₓ/λₑₘ | 4 | 1 | 3 cd | 8 cd |
| Dnp-P-L-G-L-W-A-R—NH2 | Bachem: | 280/355 | − | − | + | − |
| | M-1855 | | | | | |
| Mca-G-K—P—I-L-F—F—R-L-K-(Dnp)-R—NH2 | Sigma: | 330/390 | − | − | + | − |
| | M-0938 | | | | | |
| Mca-P-L-A-Q-A-V-Dap(Dnp)-R—S—S—S—R—NH2 | Bachem: | 330/390 | − | − | + | − |
| | M-2255/R&D | | | | | |
| | ES003 | | | | | |
| Mca-P-L-G-L-Dap(Dnp)-A-R—NH2 | Bachem: | 330/390 | − | − | + | + |
| | M-1895 | | | | | |
| Mca-P-β-cyclohexyl-A-G-Nva-H-A-Dpa-NH2 | Calbiochem: | 330/390 | − | − | + | + |
| | 444235 | | | | | |
| Mca-R—P—K—P—V-E-Nval-W—R—K-(Dnp)-NH2 | Bachem: | 330/390 | − | − | + | − |
| | M-2110/R&D | | | | | |
| | ES002 | | | | | |
| Mca-R—P-L-A-L-W—R-Dap(Dnp)-NH2 | Bachem: | 330/390 | − | − | + | + |
| | M-2390 | | | | | |
| Mca-P-L-A-C(Mob)-W-A-R-Dap(Dnp)-NH2 | Bachem: | 330/390 | − | − | + | + |
| | M-2510 | | | | | |
| Mca-R—P—K—P—Y-A-Nva-Met-K(Dnp)-NH2 | Bachem: | 330/390 | − | − | + | + |
| | M-2105 | | | | | |
| Mca-P-L-A-Nva-Dap(Dnp)-A-R—NH2 | Bachem: | 330/390 | − | − | + | + |
| | M-2520 | | | | | |
| NBD-eAhx-R—P—K—P-L-A-Nva-W—K(DMACA)-NH2 | Bachem: | 350/465 | − | − | + | + |
| | M-2300 | | | | | |
| Dnp-P-β-cyclohexyl-A-G-C(Me)-H-A-K—(N-Me-Abz)-NH2 | Bachem: | 365/450 | − | − | + | + |
| | M-2055 | | | | | |

+: Increase in the optical signal was observed over 5 minutes

As expected, and in accordance with the state of the art, the MMP substrates were not converted by ADAMTS.

### Example 2

### Preparation of the Buffers and Solutions

### TNCB Buffer (see Example 1)

### Enzyme Solution:

ADAMTS (Invitek Gesellschaft für Biotechnik & Biodesign mbH, Berlin, Germany) 5 μg of ADAMTS-1 were diluted with 2200 μL, and 5 μg of ADAMTS-4 were diluted with 600 μl of TNCB buffer.

### Substrate Solution

1) DTNB Solution (5,5′-dithiobis(2-nitrobenzoic Acid):

A 40 mM stock solution in DMSO was prepared:

Then 27.5 μL of DTNB stock solution were diluted with 522 μL of water.

### 2) Thiopeptilide Solution: (Bachem Distribution Services GmbH, Weil am Rhein, Germany):

A 100 mM stock solution in DMSO was prepared. For use, 55 μL of thiopeptilide stock solution were diluted with 500 μL of TNCB buffer.

Immediately before use, 550 μl of DTNB were mixed with 550 μl of thiopeptilide.

### Assay Procedure

The measurements were carried out in 96-multiwell plates (half area plates, flat bottom, clear, polystyrene, No. 3695) (Corning Costar, Acton, USA).

10 μL of enzyme solution and 10 μL of H₂O were mixed, and the reaction was started by adding 10 μL of substrate solution.

### Colorimetric Analysis

Microtiter plate photometer: Molecular Devices Sunnyvale, USA. SpectraMax 190.

The absorption was observed at a wavelength of 415 nm for 5 min.

### Results for Example 2

| Colorimetric substrates in | | | ADAMTS- | ADAMTS- | MMP- | MMP- |
|---|---|---|---|---|---|---|
| Ellman's reaction | | λ (nm) | 4 | 1 | 3 cd | 8 cd |
| Ac—P-L-G-[(S)-2-mercapto-4-methylpentanoyl]-L-G-OEt | Bachem: | 415 | + | + | + | + |
| | H-7145 | | | | | |
| Ac—P-L-A-[(S)-2-mercapto-pentanoyl]-W—NH2 | Bachem: | 415 | − | − | + | + |
| | H-1326 | | | | | |

| Compound 1 | Compound 2 (Comparative example |
|---|---|
| Bachem: H-7145 | Bachem: H-1326 |
| (Synonyms) | (Synonyms) |
| Ac—P-L-G-^{S}L-L-G-OEt | Ac—P-L-A-^{S}Nva-W-NH2 |
| Ac—P-L-G-[(S)-2-mercapto-4-methylpentanoyl]-L-G-OEt | Ac—P-L-A-[(S)-2-mercaptopentanoyl]-W—NH2 |
| Ac—P-L-G-Sch[CH2CH(CH3)2]-CO-L-G-OC2H5 | Ac—P-L-A-[2-mercaptopentanoyl]-W—NH2 |
| Ac—P-L-G-[2-mercapto-4-methylpentanoyl]-L-G-OC2H5 | |

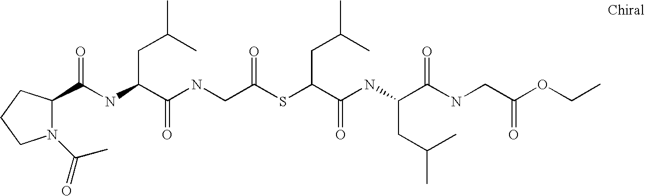

### Structural Formula of Compound 2 (Comparative Example)

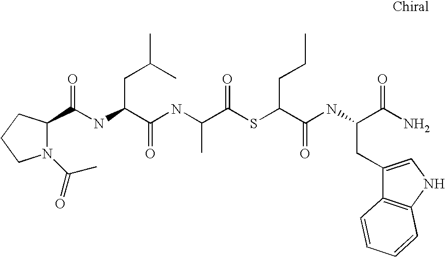

Compound 2 was not converted by ADAMTS. However, surprisingly, compound 1 was converted by ADAMTS.

| SEQ ID NO: 1 | |
|---|---|
| Xaa-Pro-Xaa-Gly-Xaa-Xaa-Gly-Xaa | |
| SEQ ID NO: 2 | |
| 1 | MTTLLWVFVT LRVITAAVTV ETSDHDNSLS VSIPQPSPLR VLLGTSLTIP CYFIDPMHPV |
| 61 | TTAPSTAPLA PRIKWSRVSK EKEVVLLVAT EGRVRVNSAY QDKVSLPNYP AIPSDATLEV |
| 121 | QSLRSNDSGV YRCEVMHGIE DSEATLEVVV KGIVFHYRAI STRYTLDFDR AQRACLQNSA |
| 181 | IIATPEQLQA AYEDGFHQCD AGWLADQTVR YPIHTPREGC YGDKDEFPGV RTYGIRDTNE |
| 241 | TYDVYCFAEE MEGEVFYATS PEKFTFQEAA NECRRLGARL ATTGHVYLAW QAGMDMCSAG |
| 301 | WLADRSVRYP ISKARPNCGG NLLGVRTVYV HANQTGYPDP SSRTDAICYT GEDFVDIPEN |
| 361 | FFGVGGEEDI TVQTVTWPDM ELPLPRNITE GEARGSVILT VKPIFEVSPS PLEPEEPFTF |
| 421 | APEIGATAFA EVENETGEAT RPWGFPTPGL GPATAFTSED LVVQVTAVPG QPHLPGGVVF |
| 481 | HYRPGPTRYS LTFEEAQQAC PGTGAVIASP EQLQAAYEAG YEQCDAGWLR DQTVRYPIVS |
| 541 | PRTPCVGDKD SSPGVRTYGV RPSTETYDVY CFVDRLEGEV FFATRLEQFT FQEALEFCES |
| 601 | HNATATTGQL YAAWSRGLDK CYAGWLADGS LRYPIVTPRP ACGGDKPGVR TVYLYPNQTG |
| 661 | LPDPLSRHHA FCFRGISAVP SPGEEEGGTP TSPSGVEEWI VTQVVPGVAA VPVEEETTAV |
| 721 | PSGETTAILE FTTEPENQTE WEPAYTPVGT SPLPGILPTW PPTGAETEES TEGPSATEVP |
| 781 | SASEEPSPSE VPFPSEEPSP SEEPFPSVRP FPSVELFPSE EPPPSKEPSP SEEPSASEEP |
| 841 | YTPSPPEPSW TELPSSGEES GAPDVSGDFT GSGDVSGHLD FSFQLSGDRA SGLPSGDLDS |
| 901 | SGLTSTVGSG LTVESGLPSG DEERIEWPST PTVGELPSGA EILEGSASGV GDLSGLPSGE |
| 961 | VLETSADGVS DLSGLPSGEV LETTAPGVED ISGLPSGEVL ETTAPGVEDI SGLPSGEVLE |
| 1021 | TTAPGVEDIS GLPSGEVLET TAPGVEDISG LPSGEVLETT APGVEDISGL PSGEVLETAA |
| 1081 | PGVEDISGLP SGEVLETAAP GVEDISGLPS GEVLETAAPG VEDISGLPSG EVLETAAPGV |
| 1141 | EDISGLPSGE VLETAAPGVE DISGLPSGEV LETAAPGVED ISGLPSGEVL ETAAPGVEDI |
| 1201 | SGLPSGEVLE TAAPGVEDIS GLPSGEVLET AAPGVEDISG LPSGEVLETA APGVEDISGL |
| 1261 | PSGEVLETAA PGVEDISGLP SGEVLETTAP GVEEISGLPS GEVLETTAPG VDEISGLPSG |
| 1321 | EVLETTAPGV EEISGLPSGE VLETSTSAVG DLSGLPSGGE VLEISVSGVE DISGLPSGEV |
| 1381 | VETSASGIED VSELPSGEGL ETSASGVEDL SRLPSGEEVL EISASGFGDL SGVPSGGEGL |
| 1441 | ETSASEVGTD LSGLPSGREG LETSASGAED LSGLPSGKED LVGSASGDLD LGKLPSGTLG |
| 1501 | SGQAPETSGL PSGFSGEYSG VDLGSGPPSG LPDFSGLPSG FPTVSLVDST LVEVVTASTA |
| 1561 | SELEGRGTIG ISGAGEISGL PSSELDISGR ASGLPSGTEL SGQASGSPDV SGEIPGLFGV |
| 1621 | SGQPSGFPDT SGETSGVTEL SGLSSGQPGV SGEASGVLYG TSQPFGITDL SGETSGVPDL |
| 1681 | SGQPSGLPGP SGATSGVPDL VSGTTSGSGE SSGITFVDTS LVEVAPTTFK EEEGLGSVEL |
| 1741 | SGLPSGEADL SGLSGMVDVS GQFSGTVDSS GFTSQTPEFS GLPSGIAEVS GESSRAEIGS |
| 1801 | SLPSGAYYGS GTPSSFPTVS LVDRTLVESV TQAPTAQEAG EGPSGILELS GAHSGAPDMS |
| 1861 | GEHSGFLDLS GLQSGLIEPS GEPPGTPYFS GDFASTTNVS GESSVAMGTS GEASGLPEVT |
| 1921 | LITSEFVEGV TEPTISQELG QRPPVTHTPQ LFRSSGLVST AGDISGATPV LPGSGVEVSS |
| 1981 | VPESSSETSA YPEAGFGASA APEASREDSG SPDLDETTSA FHEANLERSS GLGVSGSTLT |
| 2041 | FQEGEASAAP EVSGESTTTS DVGTEAPGLP SATPTASGDR TEISGDLSGH TSQLGVVIST |
| 2101 | SIPESEWTQQ TQRPAETHLE IESSSLLYSG EETHTVETAT SPTDASIPAS PEWKRESEST |
| 2161 | AAAPARSCAE EPCGAGTCKE TEGHVICLCP PGYTGEHCNI DQEVCEEGWN KYQGHCYRHF |
| 2221 | PDRETWVDAE RRCREQQSHL SSIVTPEEQE FVNNNAQDYQ WIGLNDRTIE GDFRWSDGHP |
| 2281 | MQFENWRPNQ PDNFFAAGED CVVMIWHEKG EWNDVPCNYH LPFTCKKGTV ACGEPPVVEH |
| 2341 | ARTFGQKKDR YEINSLVRYQ CTEGFVQRHM PTIRCQPSGH WEEPRITCTD ATTYKRRLQK |
| 2401 | RSSRHPRRSR PSTAH |

1-30. (canceled)

31. A method for determining the activity of an ADAM-TS protease, comprising the steps consisting of:
(a) incubating an ADAM-TS protease with a thiopeptolide substrate according to formula I
R-(Xaa)ₙ-Pro-X-Gly-S—Y-Z-Gly-(Xaa)ₘ-R₁ (formula I),
where R is H or an N-protective group, preferably a carboxyl group, in particular of C₁-C₅-alkyls, especially of C₁-C₃-alkyls, particularly preferably an acetyl group,
Xaa is any amino acid,
n, m is identically or differently an integer from 0-2415, preferably from 0-35, in particular from 0-14, especially from 0-11 and very particularly preferably equal to 0,
X is Leu, Ile, Phe, Val, Gln, Ala,
Z is Leu, Ile, Phe, Val, Gln, Ala,
R₁ is terminal amide, carboxyl or ester group, preferably of C₁-C₅-alkyls, especially of C₁-C₃-alkyls, in particular an ethyl ester, 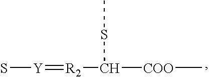
where
R₂ is the side chain of a naturally occurring amino acid, in particular
—CH₂CH(CH₃)₂,
—CH(CH₃)C₂H₅,
—CH₂C₆H₅,
—CH(CH₃)₂, or
—CH₃,
or a salt thereof,
and
(b) carrying out an activity measurement or determination of the ADAM-TS protease.

32. The method according to claim 31, characterized in that
X=Leu or Ala,
Z=Leu, Ala or Phe, and
R₂=—CH₂CH(CH₃)₂.

33. The method according to claims 31 or 32, characterized in that (Xaa)ₙ and/or (Xaa)ₘ is the amino acid sequence of SEQ ID NO: 2.

34. The method according to claim 31 wherein the thiopeptolide has the following structure:
Ac-Pro-Leu-Gly-S—Y-Leu-Gly-OC₂—H₅,
in which R₂=CH₂CH(CH₃)₂ and Ac is an acetyl group.

35. The method according to claim 31 wherein the thiopeptolide has the following structure:
Ac-Pro-Leu-Gly-S—Y-Phe-Gly-OC₂—H₅,
in which R₂=CH₂CH(CH₃)₂ and Ac is an acetyl group.

36. The method according to claim 31 wherein the thiopeptolide has the following structure:
Ac-Pro-Ala-Gly-S—Y-Phe-Gly-OC₂H₅,
in which R₂=CH₂CH(CH₃)₂ and Ac is an acetyl group.

37. The method according to claim 31 wherein the thiopeptolide has the following structure:
Ac-Pro-Ala-Gly-S—Y-Ala-Gly-OC₂—H₅,
in which R₂=CH₂CH(CH₃)₂ and Ac is an acetyl group.

38. The method according to claims 31, 32 and 34-37 in the alternative wherein said ADAM-TS protease is an ADAM-TS protease 1, 2, 3, 4, 5, 6, 7, 8, 9, 10, 11, 12, 13, 14, 15, 16, 17, 18, 19 and/or 20.

39. The method according to claims 31, 32 and 34-37 in the alternative wherein said ADAM-TS protease is selected from the group consisting of ADAM-TS protease 1, 4, 5, 11 and 13.

40. The method according to claim 31 wherein said activity of the ADAM-TS protease is measured or determined by spectrophotometry.

41. The method according to claim 40 wherein said ADAM-TS protease is measured or determined in the presence of a detection reagent for thiol groups.

42. The method according to claim 41 wherein said reagent is selected from the group consisting of iodoacetamide, maleimide, N,N′-didansyl-L-cystine, 5-(bromomethyl)fluorescein, 4,4′-dithiodipyridine and 5,5′-dithiobis(2-nitrobenzoic acid) (DTNB).

43. The method according to claim 42 wherein said iodoacetamide is 5-iodoacetamidofluorescein (5-IAF).

44. The method according to claim 42 wherein said maleimide is fluorescein-5-maleimide.

45. A method for identifying an ADAM-TS protease modulator comprising the steps consisting of:
(a) incubating an ADAM-TS protease with a thiopeptolide substrate according to formula I
R-(Xaa)ₙ-Pro-X-Gly-S—Y-Z-Gly-(Xaa)ₘ-R₁ (formula I),
where R is H or an N-protective group, preferably a carboxyl group, in particular of C₁-C₅-alkyls, especially of C₁-C₃-alkyls, particularly preferably an acetyl group,
Xaa is any amino acid,
n, m is identically or differently an integer from 0-2415, preferably from 0-35, in particular from 0-14, especially from 0-11 and very particularly preferably equal to 0,
X is Leu, Ile, Phe, Val, Gln, Ala,
Z is Leu, Ile, Phe, Val, Gln, Ala,
R₁ is terminal amide, carboxyl or ester group, preferably of C₁-C₅-alkyls, especially of C₁-C₃-alkyls, in particular an ethyl ester, 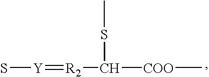
where
R₂ is the side chain of a naturally occurring amino acid, in particular
—CH₂CH(CH₃)₂,
—CH(CH₃)C₂H₅,
—CH₂C₆H₅,
—CH—(CH₃)₂, or
—CH₃,
or a salt thereof,
in the presence of a test compound and
(b) measuring or determining the influence of said test compound on the activity of the ADAM-TS protease.

46. The method according to claim 45 wherein said ADAM-TS protease is an ADAM-TS protease 1, 2, 3, 4, 5, 6, 7, 8, 9, 10, 11, 12, 13, 14, 15, 16, 17, 18, 19 and/or 20.

47. The method according to claim 46 wherein said ADAM-TS protease is selected from the group consisting of ADAM-TS protease 1, 4, 5, 11 and 13.

48. The method according to claims 45 wherein said activity of the ADAM-TS protease is measured or determined by spectrophotometry.

49. The method as claimed in claim 48, characterized in that an ADAM-TS protease is measured or determined in the presence of a detection reagent for thiol groups.

50. The method according to claim 49 wherein said reagent is selected from the group consisting of iodoacetamide, a maleimide, N,N′-didansyl-L-cystine, 5-(bromomethyl)fluorescein, 4,4′-dithiodipyridine and 5,5′-dithiobis(2-nitrobenzoic acid (DTNB).

51. The method according to claim 50 wherein said iodoacetamide is 5-iodoacetamidofluorescein (5-IAF).

52. The method according to claim 50 wherein said maleimide is fluorescein-5-maleimide.

53. The method according to claims 45-52 in the alternative wherein said test compound is made available in the form of a chemical compound library.

54. The method according to claims 45-52 in the alternative wherein said method is carried out on an array.

55. The method according to claim 45 wherein said method is carried out by means of a robot.

56. The method according to claim 45 wherein said method is carried out with the aid of microfluidic technology.

57. The method according to claim 45 wherein said method is a high-throughput screening for an ADAM-TS protease inhibitor.

58. A kit comprising a thiopeptolide substrate according to formula I
R-(Xaa)ₙ-Pro-X-Gly-S—Y-Z-Gly-(Xaa)ₘ-R₁ (formula I),
where R is H or an N-protective group, preferably a carboxyl group, in particular of C₁-C₅-alkyls, especially of C₁-C₃-alkyls, particularly preferably an acetyl group,
Xaa is any amino acid,
n, m is identically or differently an integer from 0-2415, preferably from 0-35, in particular from 0-14, especially from 0-11 and very particularly preferably equal to 0,
X is Leu, Ile, Phe, Val, Gln, Ala,
Z is Leu, Ile, Phe, Val, Gln, Ala,
R₁ is terminal amide, carboxyl or ester group, preferably of C₁-C₅-alkyls, especially of C₁-C₃-alkyls, in particular an ethyl ester, 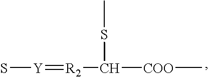
where
R₂ is the side chain of a naturally occurring amino acid, in particular
—CH₂CH(CH₃)₂,
—CH(CH₃)C₂H₅,
—CH₂C₆H₅,
—CH(CH₃)₂, or
—CH₃,
or a salt thereof,
an ADAM-TS protease and where appropriate one or more buffers.

59. The kit according to claim 58, wherein said ADAM-TS protease is an ADAM-TS protease 1, 2, 3, 4, 5, 6, 7, 8, 9, 10, 11, 12, 13, 14, 15, 16, 17, 18, 19 and/or 20.

60. The kit according to claim 58 wherein said ADAM-TS protease is an ADAM-TS protease 1, 4, 5, 11 and/or 13.

61. The kit according to claim 58 wherein a detection reagent for thio groups is additionally present.

62. The kit according to claim 61 wherein said reagent is selected from the group consisting of iodoacetamide, maleimide, N,N′-didansyl-L-cystine, 5-(bromoethyl)fluorescein, 4,4′-dithiodipyridine, 5,5′-dithiobis(2-nitrobenzoic acid) (DTNB).
